(19) United States
(12) Patent Application Publication
(10) Pub. No.: US 2022/0333196 A1
(43) Pub. Date: Oct. 20, 2022

(54) Methods and Systems for Labelling Nucleic Acids

(71) Applicant: IMEC VZW, Leuven (BE)

(72) Inventor: Dries Braeken, Wilsele (BE)

(21) Appl. No.: 17/720,005

(22) Filed: Apr. 13, 2022

(30) Foreign Application Priority Data
Apr. 14, 2021 (EP) 21168293.5

(51) Int. Cl. C12Q 1/6881; C12N 13/00

(52) U.S. Cl. CPC C12Q 1/6881; C12N 13/00

(57) ABSTRACT

Systems and methods are provided for labelling nucleic acids in a cell of a cell culture or of a tissue. The systems and methods include contacting the cell culture or tissue with a first label and applying an electrical field to the cell, thereby increasing the permeability of the cell membrane, allowing the first label to be introduced into the cell. The cell culture or tissue can be disposed on a multielectrode array, and the electrical field can be applied by operating one or more electrodes of the array that are proximate to (e.g., beneath) the cell in order to control the location of the cell whose permeability is increased and/or to control the timing of such permeability increase.

### CROSS-REFERENCE TO RELATED APPLICATIONS

The present application is a non-provisional patent application claiming priority to European Patent Application No. EP 21168293.5, filed Apr. 14, 2021, the contents of which are hereby incorporated by reference.

### FIELD OF THE DISCLOSURE

The present invention relates to the field of nucleic acid labelling (e.g., barcoding), and more particularly to the field of single-cell nucleic acid sequencing, such as transcriptome sequencing.

### BACKGROUND

In precision medicine, medical decisions, treatments, practices, or products are tailored to a subgroup of patients, instead of using a one-drug-fits-all model. Cell transcriptome RNA sequencing has become a very powerful technique in that field. It is the gold standard for defining cell states and phenotypes. It allows examining the expression profiles of genes of interest by sequencing RNA molecules present in cells of the patient. Typically, cells (e.g., fibroblasts or blood cells) are taken out of the patient and are cultured on a chip. Eventually, these cells may be reprogrammed to become different types of cells, such as neurons. This is especially useful since the brain of a live patient is typically not accessible for neural tissue extraction or detailed investigation in situ. The transcriptome of these cells can then be acquired. Typically, this is done in bulk, i.e., on a large number of cells originating in a single patient but lumped together. This means that at a certain moment, the cell culture is stopped, the cells are harvested and mixed, their RNA is taken out and sequenced. As a result, the transcriptome obtained is statistical data representing a large number of cells. Single-cell isolation remains difficult and needs cell detachment from the neighboring cells in the cell culture. Although this technique allows you to group certain cells by phenotype, it is not possible to trace back which phenotype was expressed by which cell in the cell culture.

RNA sequencing methods depend on poly-A tail capture to enrich mRNA and deplete abundant and uninformative rRNA, although other types of RNA can also be captured today. Spatial transcriptomics resolves RNA sequence data in individual tissue sections or cell cultures. This is done by attaching barcoded oligo(dT) primers at various specific locations of the surface of a microscope slide to encode the positional information after sequencing. Since different positions are barcoded differently, spatial information can be retrieved after sequencing. A challenge here is to obtain a high density of primers, and the difficulty to capture efficiently RNA from the cell culture or tissue above.

Acquiring transcriptome information at a single-cell resolution is today not possible since most transcriptome analysis is done in bulk. With high-resolution spatial transcriptomics, one could achieve single cell or even subcellular resolution, but the drawback is that you need specific slides with barcodes pre-printed on them or you need to use beads or other vehicles. Also, the method is not very versatile. In principle, one can take out the mRNA from single cells by patch pipettes (Patch sequencing), and then perform sequencing. This method is, however, not very scalable.

For all cells, but in particular, for cardiac cells and neurons, an electrophysiological read-out of the state of the cells of which the transcriptome is obtained is of great importance. This permits linking the electrophysiology of the cell to its expression profile. However, acquiring a combination of electrophysiological and transcriptome information at a single-cell resolution is today not possible.

Also, current techniques only give the transcriptome at a given time. How that transcriptome evolves in time is not accessible.

Similar issues exist with cancer biopsies where characterizing the heterogeneity of cancer cell genomes is important. For such applications, both mRNA and DNA sequencing are relevant. An additional issue in that field is that it is not currently possible to sequence DNA and mRNA at the same time while simultaneously retaining where these DNAs and mRNAs were spatially located.

There is therefore a need in the art for new methods and systems overcoming at least partially one or more of these issues.

### SUMMARY

It is an object of the present disclosure to provide systems or methods for labelling (e.g. barcoding) nucleic acid in a cell.

The above objective is accomplished by a method and device according to the present disclosure.

In a first aspect, a method is provided for labelling (e.g. barcoding) nucleic acids in a cell of a cell culture or of a tissue, the method comprising:

a) contacting the cell culture or tissue with a first label (e.g., a barcoded nucleic acid-capturing probe) for labelling nucleic acid molecules, and

b) applying an electrical field to the cell, thereby increasing the permeability of the cell membrane, allowing the first label to be introduced into the cell.

In a second aspect, a system is provided for labelling (e.g., barcoding) nucleic acid in a cell of a cell culture or of a tissue, the system comprising a multi electrode array (MEA) having a density of electrodes equal to or higher than 1000 electrodes per square millimeter, each electrode (3) being addressable individually for applying an electric field.

wherein the system is configured to bring a label (e.g., a barcoded nucleic acid-capturing probe) in contact with a cell when a cell culture is present on the chip.

It is a benefit of some of the embodiments described herein that single specific cells can be labelled (e.g., barcoded). This label (e.g., barcode) can record the exact position of the cell in the cell culture, provide a timestamp, or both.

It is a benefit of some of the embodiments of the present disclosure that the transcriptome of single specific cells can be obtained instead of obtaining a statistical transcriptome lumping data of a plurality of cells. The exact position of that specific cell and, in some embodiments, the exact time at which the cell has been labelled (e.g., barcoded), is recorded by the labelling (e.g. barcoding).

It is a benefit of some of the embodiments of the present disclosure that the transcriptome of single specific cells can be obtained without having to destroy the cell culture or tissue in which the cell is located.

It is a benefit of some of the embodiments of the present disclosure that single specific cells and even portions of specific cells can be studied. For instance, their electrophysiological data and/or their transcriptome can be acquired, and in some embodiments, followed in time.

It is a benefit of some of the embodiments of the present disclosure that one does not need elaborate techniques to keep track of where in a cell culture a nucleic acid of interest originates from. Indeed, that information is attached to the retrieved nucleic acid (typically mRNA, DNA, or both) itself by virtue of the labelling (e.g., barcoding).

It is a benefit of some of the embodiments of the present disclosure that electrophysiological data and gene expression information can be acquired simultaneously with a single-cell resolution or even with a subcellular resolution.

It is a benefit of some of the embodiments of the present disclosure that expensive slides with pre-printed barcoded probes are not necessary.

It is a benefit of some of the embodiments of the present disclosure that no extracellular vehicles such as beads are needed.

It is a benefit of some of the embodiments of the present disclosure that they are easily scalable.

It is a benefit of some of the embodiments described herein that they facilitate every cell of a cell culture or of a tissue being quickly barcoded with information unambiguously identifying its position in the culture.

It is a benefit of some of the embodiments described herein that they allow monitoring of gene expression over time at a single cell level following a stimulus.

It is a benefit of some of the systems of the present disclosure that they are also suitable for introducing elements (e.g., transcription factors) in one or more specific stem cells that would enable their transformation in a differentiated cell such as a neuron or a cardiac cell.

It is a benefit of some of the embodiments of the present disclosure that they make precision medicine more easily achieved, more precise, and more affordable.

Particular and example embodiments are set out in the accompanying independent and dependent claims. Features from the dependent claims may be combined with features of the independent claims and with features of other dependent claims as appropriate and not merely as explicitly set out in the claims.

Although there has been constant improvement, change, and evolution of devices in this field, the present concepts are believed to represent substantial new and novel improvements, including departures from prior practices, resulting in the provision of more efficient, stable, and reliable devices of this nature.

The above and other characteristics, features, and advantages of the presently disclosed embodiments will become apparent from the following detailed description, taken in conjunction with the accompanying drawings, which illustrate, by way of example, the principles of the subject matter described herein. This description is given for the sake of example only, without limiting the scope of the claims. The reference figures quoted below refer to the attached drawings.

### BRIEF DESCRIPTION OF THE FIGURES

The above, as well as additional, features will be better understood through the following illustrative and non-limiting detailed description of example embodiments, with reference to the appended drawings.

FIG. 1 is a flowchart depicting an example process, according to an example embodiment.

FIG. 2 is a schematic representation of a multielectrode array, according to an example embodiment.

FIG. 3 is a schematic representation of a multielectrode array sub-unit, according to an example embodiment.

FIG. 4 is a schematic representation of aspects of a method, according to an example embodiment.

All the figures are schematic, not necessarily to scale, and generally only show parts which are necessary to elucidate example embodiments, wherein other parts may be omitted or merely suggested.

### DETAILED DESCRIPTION

The subject matter described herein will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to specific actual reductions to practice.

Furthermore, the terms first, second, third, and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments described herein are capable of operation in other sequences than described or illustrated herein.

Moreover, the terms top, bottom, over, under, and the like in the description and the claims are used for descriptive purposes and not necessarily for describing relative positions. It is to be understood that the terms so used are interchangeable under appropriate circumstances and that the embodiments described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps, or components as referred to, but does not preclude the presence or addition of one or more other features, integers, steps or components, or groups thereof. The term “comprising” therefore covers the situation where only the stated features are present (and can therefore always be replaced by “consisting of” in order to restrict the scope to the stated features) and the situation where these features and one or more other features are present. The word “comprising” therefore also includes as one embodiment that no further components are present. Thus, the scope of the expression “a device comprising means A and B” should not be interpreted as being limited to devices consisting only of components A and B. It means that with respect to the present disclosure, the only relevant components of the device are A and B.

Reference throughout this specification to “one embodiment” or “an embodiment” means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures, or characteristics may be combined in any suitable manner, as would be apparent to one of ordinary skill in the art from this disclosure, in one or more embodiments.

Similarly, it should be appreciated that in the description of exemplary embodiments, various features are sometimes grouped together in a single embodiment, figure, or description thereof for the purpose of streamlining the disclosure and aiding in the understanding of one or more of the various aspects described herein. This method of disclosure, however, is not to be interpreted as reflecting an intention that the claimed subject matter requires more features than are expressly recited in each claim. Rather, as the following claims reflect, aspects of the present disclosure lie in less than all features of a single foregoing disclosed embodiment. Thus, the claims following the detailed description are hereby expressly incorporated into this detailed description, with each claim standing on its own as a separate embodiment.

Furthermore, while some embodiments described herein include some but not other features included in other embodiments, combinations of features of different embodiments are meant to be within the scope of the claims, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the subject matter described herein.

In the description provided herein, numerous specific details are set forth. However, it is understood that embodiments described herein may be practiced without these specific details. In other instances, well-known methods, structures, and techniques have not been shown in detail in order not to obscure an understanding of this description.

The following terms are provided solely to aid in the understanding of the subject matter described herein.

As used herein, and unless provided otherwise, the term “labelling” refers to the action of providing nucleic acid molecules, such as mRNA or DNA molecules, with a particular label. The label is typically a chemical moiety, or a molecule, suitable to serve as an identifier. The label can either attach to the nucleic acid molecule or can be incorporated in the nucleic acid molecule, e.g., during its synthesis. An example where the label is attached to the nucleic acid molecule is when the nucleic acid molecule is labelled with a barcoded nucleic acid-capturing; probe. An example where the label is incorporated in the nucleic acid molecule is when the nucleic acid molecule is synthesized in presence of a metabolic label which will get incorporated in the nucleic acid molecule sequence.

As used herein, and unless provided otherwise, the term “barcoding” refers to the action of labelling nucleic acid molecules, such as mRNA or DNA molecules, with a particular barcoded nucleic acid-capturing probe. The barcoded nucleic acid-capturing probe is typically a molecule capable of hybridizing with the nucleic acid molecule and suitable to serve as an identifier. For barcoding mRNA, a barcoded nucleic acid-capturing probe can be a single strand polynucleotide (e.g. DNA) comprising an oligo(dT) sequence for hybridizing with the poly(A) tail of mRNA, a PCR handle sequence for enabling PCR amplification, and a barcode sequence suitable to serve as an identifier of either the location of the cell, the time of transfection, or both. For DNA barcoding, for example, labelled single-strand DNA or oligonucleotides can be used that hybridize to a specific DNA sequence.

As used herein, and unless provided otherwise, a metabolic label is a molecule that can be incorporated in a nucleic acid molecule sequence doting its synthesis, typically rendering that nucleic acid molecule noncanonical. This molecule can, for instance, be a noncanonical nucleoside or nucleotide. Once incorporated in the nucleic acid molecule, the nucleic acid molecule can be identified by detecting the presence of the label within the sequence of the nucleic acid molecule. For instance, the label can be detected as a mutation of the nucleic acid molecule. One way to perform this is described in J. A. Schofield et al., Nature methods, vol. 15 No. 3, March 2018, 221-225.

A detailed description of several example embodiments will now be provided. It is clear that other embodiments can be configured according to the knowledge of persons skilled in the art without departing from the technical teaching of the present disclosure, the scope of the claimed subject matter being limited only by the terms of the appended claims.

In a first aspect, a method is provided for labelling (e.g., barcoding) nucleic acids in a cell of a cell culture or of a tissue.

The cell is a biological cell. It is in some examples a eukaryotic cell. For example, it can be an animal cell. For example, it can be a mammalian cell. For example, it can be a human cell. For example, it can be a neuron. The embodiments described herein are well suited for the field of precision medicine, where human cells are involved. The embodiments described herein are well suited for labelling (e.g., barcoding) and simultaneously recording the electrochemical activity of neurons.

How to form a cell culture on a substrate is well known to the person skilled in the art. One way to obtain a particular cell culture of animal or human cells only extractable from a body by surgery (such as neurons) is to first form a cell culture of body cells extractable from a body without surgery (such as fibroblasts or blood cells), then reprogramming the cell culture to become the cell culture of animal or human cells only extractable from a body by surgery (such as neurons or muscle cells). How to do so is well known to the person skilled in the art.

Tissues can be analyzed as well by the methods described herein. For instance, the cell can be of a biopsy or of an organoid.

The nucleic acids may be DNA or RNA. If they are RNA, they can be, e.g., mRNA. mRNA present in a cell gives information on the type of genes currently active in that cell. The full range of messenger RNA, or mRNA, molecules expressed by a cell is called its transcriptome. The embodiments described herein are useful for obtaining a transcriptome of a cell or of a cell culture or of a tissue. If they are DNA, the embodiments described herein are useful for determining the genome of a plurality of cells within a cell culture or tissue. This allows the characterization of the heterogeneity of the culture or tissue. This has applications in the field of cancer biopsies where characterizing the heterogeneity of cancer cell genomes is important.

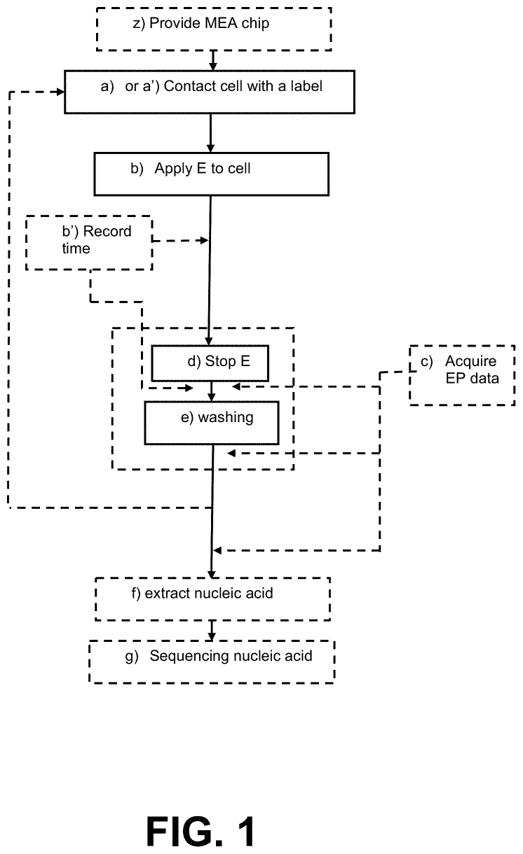

We now refer to FIG. 1. Dashed rectangles indicate optional steps. Plain-lined rectangles indicate mandatory steps.

Typically, the method described herein includes the presence of at least one electrode (3) configured to apply an electrical field to a cell (2) of a cell culture or of a tissue. The at least one electrode (3) can also be configured, in some embodiments, to measure electrical signals from the cell (2).

In the example embodiment depicted, a plurality of electrodes (3) is used, wherein each electrode (3) is below a different part of the cell culture or tissue. It can be beneficial to configure each electrode (3) within the plurality to be addressable individually to apply the electrical field. For example, each electrode (3) within the plurality could be addressable individually to apply the electrical field and to measure electrical signals from the cell (2).

Typically, at least one electrode (3) having a cell culture thereon is provided in a step z) performed before step a).

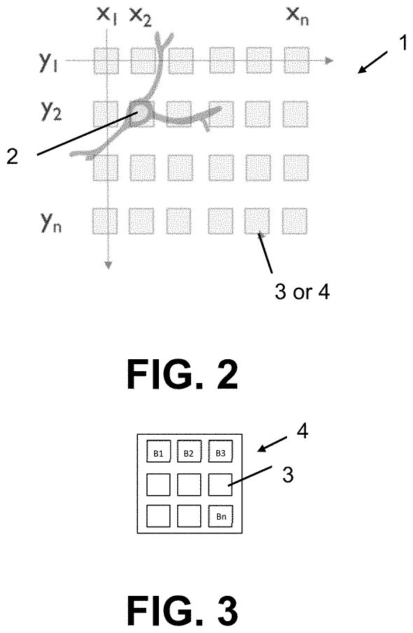

Step z) can include providing a multielectrode array (1) having a cell culture thereon, wherein a density of electrodes (3) in the multielectrode array (1) is equal to or higher than a density of cells (2) in the cell culture or tissue, wherein applying an electrical field to the cell (2) in step b) is performed by means of an electrode (3) present under the cell (2). An example of such a multielectrode array (1) is depicted in FIG. 2. This multielectrode array (1) is depicted as having Xn columns of pixels (4) and Yn rows of pixels (4). A neuron is depicted above one of the pixels. The pixel can be one electrode (3) or a plurality of electrodes (3). FIG. 3 shows one pixel being composed of a plurality (Bn) of electrodes (3). The use of a density of electrodes (3) which is smaller or equal to the density of cells (2) is beneficial because it permits to address (and optionally record) each cell (2) individually.

In embodiments, the density of electrodes (3) may be equal to or higher than 1000 electrodes per square millimeter, for example from 1000 to 20000 electrodes per millimeter square, from 1000 to 10000 electrodes per millimeter square, or from 5000 to 15000 electrodes per millimeter square.

In some example embodiments, the multielectrode array (1) may comprise:

an active sensor area presenting a surface for cell (2) growth on the device;

a microelectrode array (1) comprising a plurality of pixels (4) in the active sensor area, wherein each pixel comprises at least one electrode (3) at the surface, wherein the at least one electrode (3) is configured to form contact with cells (2) for providing stimulating signals to cells (2) and/or measuring electrical signals from cells (2), wherein each pixel further comprises pixel circuitry comprising at least one switch for setting a configuration of the pixel circuitry and wherein each pixel is configured to individually receive a control signal for controlling the configuration of the pixel circuitry and set a measurement modality of the pixel;

recording circuitry having a plurality of recording channels, wherein each pixel is connected to a recording channel, the recording channel being configured to receive signals from the pixels (4) in the active sensor area, and

wherein each recording channel of the recording circuitry comprises a reconfigurable component, which is selectively controlled between being set to a first mode, in which the reconfigurable component is configured to amplify a received pixel signal, and being set to a second mode, in which the reconfigurable component is configured to selectively pass a frequency band of the received pixel signal. An example of a multielectrode array (1) usable in the methods described herein is described in US2020018742, the content of which is incorporated herein in its entirety.

The first step of the method, i.e., step a), comprises contacting e cell culture or tissue with a first label (e.g., a barcoded nucleic acid capturing probe) (5) for labelling nucleic acids.

In embodiments, step a) may comprise providing a fluid comprising the first label (e.g. barcoded nucleic acid-capturing, probe) (5) and contacting the fluid with the cell culture or tissue. The fluid is typically a liquid. This can be done in many ways. One way is to drop the fluid on the cell culture or tissue. Another way is to bring the fluid in contact with the cell culture or tissue by means of a fluidic channel fluidically connected with the cell culture. In embodiments, the fluidic channel can form part of a microfluidic system. Microfluidic systems have the benefit of permitting the delivery of the label in high concentration. It also has the benefit of permitting very good flushing and automation. A control unit can be coupled to the microfluidic system. The control unit and the microfluidic system can be configured to perform the steps of the method.

In embodiments, the fluidic channel can be transparent. This has the benefit of permitting observation of its content.

Step b) of the method comprises applying an electrical field to the cell (2), thereby increasing the permeability of the cell (2) membrane, allowing the first label (e.g. barcoded nucleic acid-capturing probe) (5) to be introduced into the cell (2). Step b) is typically performed by means of an electrode (3) present under the cell (2).

Step b) allows the label (e.g., barcoded nucleic acid capturing probe) (5) to enter the cell (2) and to label (e.g. hybridize with the nucleic acid present in the cell (2) or being incorporated in the sequence of a growing nucleic acid). Typically, step b) is performed for a time sufficient to allow labelling (e.g. hybridization) of the nucleic acid present in the cell (2) with the probe (5). Once this time has passed, the electrical field is typically interrupted.

In some example embodiments, the method may comprise a step c) of acquiring electrophysiological data about the cell (2), wherein the method is for labelling (e.g., barcoding) nucleic acids in the cell (2) and for obtaining an electrophysiological signature of the cell (2). Typically, step c) is performed by means of the electrode (3) present under the cell (2). The same electrode (3) can, therefore, apply the electrical field and obtain an electrophysiological signature of the cell (2).

In embodiments, the method may further comprise a step d) of stopping the electrical field, a step e) of washing off any label (e.g. barcoded nucleic acid-capturing probe or noncanonical nucleoside or nucleotide) (5) present outside of the cell (2) and in contact with the cell culture or tissue, a step a′) of contacting the cell culture or tissue with a further label (e.g., barcoded nucleic acid-capturing probe) (5), different from any label (e.g., barcoded nucleic acid-capturing probe) (5) previously used to contact the cell culture or tissue in previous steps of the method, and a further step b), thereby increasing the permeability of the cell (2) membrane, allowing the further label (e.g., barcoded nucleic acid-capturing probe) (5) to be introduced into the cell (2). The further label (e.g., barcoded nucleic acid-capturing probe) (5) is different from the first label (e.g. barcoded nucleic acid-capturing probe) (5) (and from any label (5) previously used to contact the cell culture or tissue in previous steps) so that it can serve as a further identifier of the cell (2) location or as an identifier of a time of transfection, or both. Typically, when the label and the further label are barcoded nucleic acid-capturing probes (5), they differ at the level of their barcode sequences. When the label and the further label are metabolic labels, the labels may differ in their chemical structures.

For instance, the first label (e.g., barcoded nucleic acid-capturing probe) (5) can serve as an indication of the location of the cell (2), such as above which row and which column of the multielectrode array (1) the cell (2) is, and the second label (e.g., a barcoded nucleic acid-capturing probe) (5) can serve as an indication of the time at which the second label (e.g., barcoded nucleic acid capturing probe) has been transfected in the cell (2). The knowledge of this time can be beneficial, for instance, if an electrophysiological measurement was performed at that time. It is also useful to know at what time was the cell (2) having a particular gene expression profile, as determined by its transcriptome.

As another example, the first label (e.g., barcoded nucleic acid-capturing probe) (5) can serve as a first indication of the location of the cell (2), such as above which column or row of the multielectrode array (1) the cell (2) is, and the second label (e.g., barcoded nucleic acid-capturing probe) (5) can serve as a second indication of the location of the cell (2), such as, respectively, above which row or column of the multielectrode array (1) the cell (2) is. Using a same first label (e.g., barcoded nucleic acid-capturing probe) (5) for each cell (2) above a same column of a multielectrode array (1), and using a same second label (e.g., barcoded nucleic acid-capturing probe) (5), different from the first label (barcoded nucleic acid-capturing probe) (5), for each cell (2) above a same row of a multielectrode array (1), enable the use of a limited number of labels (e.g., barcoded nucleic acid-capturing probes) (5) to specify the location of each cell (2) above the array (1). The alternative of using a different label (e.g., barcoded nucleic acid-capturing probe) (5) for each row-column combination is also possible but requires many more different labels (e.g., barcoded nucleic acid-capturing probes) (5).

Hence, in embodiments, when a multielectrode array (1) is used, the array (1) may comprise a plurality of columns and rows of electrodes (3), wherein step b) is performed simultaneously on a plurality of cells (2) by means of a plurality of electrodes (3) of a same column or row, thereby increasing the permeability of the cell (2) membranes of the plurality of cells (2), thereby allowing a same label (e.g., barcoded nucleic acid-capturing probe) (5) to be introduced into the plurality of cells (2). This has the benefit of limiting the number of times step b) has to be performed. This also has the benefit of limiting the number of different labels (barcoded nucleic acid-capturing probes) (5) that have to be used. In such embodiments, step b) may be repeated for each column and each row of the array (1), wherein electrodes (3) of a same column or row is introduced with a same label (e.g., barcoded nucleic acid-capturing probe) (5), wherein electrodes (3) of different columns or rows are introduced with different labels (e.g., barcoded nucleic acid-capturing probes) (5).

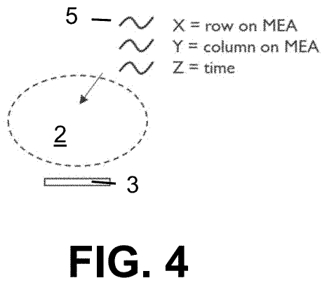

In embodiments, steps d), e), a′), and b) can be performed one or more additional times. This means that three or more labels (e.g., barcoded nucleic acid-capturing probes) (5) are used to tag a same cell (2). This typically enables to specify the location of the label (e.g. probe) (5) and at least one transfection time. This is depicted in FIG. 4. Typically, once three different labels (e.g., barcoded nucleic acid-capturing probes) (5) have been used, any further label (barcoded nucleic acid-capturing probe) (5) used will serve as a timestamp. This allows seeing how the electrophysiological state of the cell (2) and/or its transcriptome evolves over time.

In embodiments, instead of transfecting a same cell (2) repeatedly with different labels (5), it can be useful to transfect different but neighboring cells (2) with consecutive different labels (5). Although this method loses in spatial resolution, it may sometimes allow the use of more different labels (5), thereby allowing a temporal analysis of neighboring cells (2) with more time points than would be possible on a single cell (2). In such embodiments, the use of a multielectrode array (1) wherein each pixel is composed of a plurality of electrodes (3), as shown in FIG. 3, and wherein each electrode (3) is below a different cell (2), allows such a multi-time points temporal analysis. For instance, electrode (3) B1 can be used to transfect a label (5) that will serve to mark the column where the pixel is, electrode (3) B2 can be used to transfect a label (5) that will serve to mark the row where the pixel is, electrode (3) B3 can be used to transfect a label (5) that will serve as a first time stamp, and electrode (3) Bn can be used to transfect a label (5) that will serve as an n^{th-2} time stamp.

In embodiments, the method may further comprise a step b′) of recording the time at which a label (e.g., a barcoded nucleic acid-capturing probe) (5) is allowed to enter into the cell (2). This permits associating a time-stamp to that label (e.g. barcode).

In embodiments, two successive different labels (e.g., barcoded nucleic acid capturing probes) (5) may be allowed to enter into the cell (2) separated by a time of at least 30 min. This permits determining the state of the cell (2) (e.g., transcriptome or electrophysiological state) at two different times separated by at least 30 min.

In embodiments, the method may further comprise f) extracting nucleic acid molecules from the cell (2), and g) sequencing the nucleic acid molecules. Typically, between step f) and step g), a step of separating the nucleic acid (e.g., mRNA) molecules from other cellular components, and a step of enriching the nucleic acid (e.g., mRNA) molecules will also be performed.

In a second aspect, a system is provided for labelling (e.g., barcoding) nucleic acids in a cell (2) of a cell culture or of a tissue, the system comprising a multielectrode array (1) having a density of electrodes (3) equal or higher than 1000 electrodes per millimeter square, each electrode (3) being addressable individually for applying an electric field.

wherein the system is configured to bring a label (e.g., a barcoded nucleic acid-capturing probe) (5) in contact with a cell (2) when a cell culture is present on the chip.

Any feature of the second aspect may be as correspondingly described for the first aspect. For instance, as indicated in the first aspect,

In some example embodiments, the multielectrode array (1) may comprise:
an active sensor area presenting a surface for cell (2) growth on the device;
a microelectrode array (1) comprising a plurality of pixels (4) in the active sensor area, wherein each pixel (4) comprises at least one electrode (3) at the surface, wherein the at least one electrode (3) is configured to form contact with cells (2) for providing stimulating signals to cells (2) and/or measuring electrical signals from cells (2), wherein each pixel (4) further comprises pixel (4) circuitry comprising at least one switch for setting a configuration of the pixel (4) circuitry and wherein each pixel (4) is configured to individually receive a control signal for controlling the configuration of the pixel (4) circuitry and set a measurement modality of the pixel (4);
recording circuitry having a plurality of recording channels, wherein each pixel (4) is connected to a recording channel, the recording channel being configured to receive signals from the pixels (4) in the active sensor area, and

wherein each recording channel of the recording circuitry comprises a reconfigurable component, which is selectively controlled between being set to a first mode, in which the reconfigurable component is configured to amplify a received pixel (4) signal, and being set to a second mode, in which the reconfigurable component is configured to selectively pass a frequency band of the received pixel (4) signal. An example of a multielectrode array (1) usable in the methods described herein is described in US2020018742, the content of which is incorporated herein in its entirety.

Example:

First, an MEA is provided having a culture of cells (2) thereon. This is depicted as step z) in FIG. 1. This is also schematized in FIG. 2.

Second, the cell culture is contacted with a first label (e.g., a barcoded nucleic acid-capturing probe) (5) present in a liquid solution. This is depicted as step a) in FIG. 1.

Third, an electrical pulse train is applied to a first column X1 of the array (1), thereby increasing the permeability of the cell (2) membrane into each cell (2) above the first column, allowing the first label (e.g., barcoded nucleic acid-capturing probe) (5) to be introduced into each cell (2) above the first column. This is represented by step b) in FIG. 1. A certain time (e.g., one minute) is then allowed to pass to give the first label (e.g., barcoded nucleic acid-capturing probe) (5) the time to enter the cell (2) and bind to the mRNA inside the cell (2).

Fourth, the electrical pulse train is stopped (step d in FIG. 1).

Fifth, any label (e.g., barcoded nucleic acid-capturing probe) (5) outside of the cell (2) and in contact with the cell culture is washed off. This is depicted in step e) of FIG. 1.

Sixth, the process going from the second step to the fifth step is repeated for each remaining column (X2 . . . Xn) and for each row of the array (1) (Y1 . . . Yn), each time using a different barcoded label (e.g., nucleic acid-capturing probe) (5).

Seventh, the process going from the second step to the sixth step is repeated one or more times, but now the time is recorded (step b′ in FIG. 1) somewhere between step b) and step e) and electrophysiological data (step c in FIG. 1) is acquired somewhere between step d) and either step f) or a further step a).

Eight, the cells (2) are collected and lysed (step f in FIG. 1), the mRNA is extracted and sequenced (step g in FIG. 1)

It is to be understood that although example embodiments, specific constructions, and configurations, as well as materials, have been discussed herein for devices as described herein, various changes or modifications in form and detail may be made. For example, any formulas given above are merely representative of procedures that may be used. Functionality may be added or deleted from the block diagrams and operations may be interchanged among functional blocks. Steps may be added or deleted to methods as described herein.

In the above embodiments have mainly been described with reference to a limited number of examples. However, as is readily appreciated by a person skilled in the art, other examples than the ones disclosed above are equally possible within the scope of the embodiments described herein, as defined by the appended claims

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A method for labelling nucleic acids in a cell of a cell culture or tissue, the method comprising:
contacting the cell culture or tissue with a first label for labelling nucleic acids; and
applying an electrical field to the cell, thereby increasing a permeability of a cell membrane of the cell, thereby allowing the first label to be introduced into the cell.

2. The method of claim 1, further comprising:
before contacting the cell culture or tissue with the first label, providing a multielectrode array having the cell culture or tissue disposed thereon, wherein a density of electrodes of the multielectrode array is equal to or higher than a density of cells in the cell culture or tissue, wherein applying an electrical field to the cell comprises applying the electrical field via an electrode of the multielectrode array that is located under the cell.

3. The method of claim 1, wherein the cell is a neuron.

4. The method of claim 1, wherein the label is at least one of a barcoded nucleic acid capturing probe or a metabolic label.

5. The method of claim 1, further comprising:
acquiring electrophysiological data about the cell.

6. The method of claim 5, further comprising:
before contacting the cell culture or tissue with the first label, providing a multielectrode array having the cell culture or tissue disposed thereon, wherein a density of electrodes of the multielectrode array is equal to or higher than a density of cells in the cell culture or tissue, wherein applying an electrical field to the cell comprises applying the electrical field via an electrode of the multielectrode array that is located under the cell, and wherein acquiring electrophysiological data about the cell comprises acquiring electrophysiological data about the cell via the electrode that is located under the cell.

7. The method according to claim 1, further comprising:
subsequent to applying the electrical field, washing off an amount of the first label that is present outside of the cell and in contact with the cell culture or tissue;
contacting the cell culture or tissue with a second label, wherein the second label differs from any label previously used to contact the cell culture; and
applying an electrical field to the cell a second time, thereby increasing the permeability of the cell membrane, thereby allowing the second label to be introduced into the cell.

8. The method according to claim 7, further comprising:
subsequent to applying the electrical field the second time, washing off an amount of the second label that is present outside of the cell and in contact with the cell culture or tissue;
contacting the cell culture or tissue with a third label, wherein the third label differs from any label previously used to contact the cell culture; and
applying an electrical field to the cell a third time, thereby increasing the permeability of the cell membrane, thereby allowing the third label to be introduced into the cell.

9. The method according to claim 1, further comprising:
recording a time at which the first label is allowed to be introduced into the cell.

10. The method according to claim 2, wherein the array comprises a plurality of columns and rows of electrodes, and wherein the method further comprises:
while applying an electrical field to the cell via the electrode, applying an electrical field to a plurality of additional cells via a plurality of additional electrodes of the multielectrode array that are part of a same column or row, thereby increasing a permeability of cell membranes of the plurality of additional cells, thereby allowing the first label to be introduced into the plurality of additional cells.

11. The method according to claim 10, wherein applying electrical fields to cells of the cell culture or tissue via electrodes of the multielectrode array is repeated for each column and each row of the array, thereby introducing into cells of the cell culture or tissue that are proximate to electrodes of a same column or row is introduced a same label and introducing into cells of the cell culture or tissue that are proximate to electrodes of different columns or rows different labels.

12. The method according to claim 1, wherein contacting the cell culture or tissue with the first label comprises providing a fluid comprising the first label and contacting the fluid with the cell culture.

13. The method according to claim 1, further comprising:
extracting nucleic acid molecules from the cell; and
sequencing the nucleic acid molecules.

14. The method according to claim 13, further comprising:
obtaining a transcriptome of the cell of the cell culture or tissue based on sequencing the nucleic acid molecules.

15. A system for labelling nucleic acids in a cell of a cell culture or of a tissue, the system comprising a multielectrode array having a density of electrodes equal to or higher than 1000 electrodes per millimeter square, each electrode being addressable individually for applying an electric field,
wherein the system is configured to bring a label in contact with a cell when a cell culture is present on the chip.

16. The system of claim 15, further comprising:
circuitry comprising at least one switch for setting a configuration of at least one pixel of the multielectrode array, thereby setting a measurement modality of at least one electrode of the multielectrode array that is part of the at least one pixel.

17. The system of claim 16, further comprising recording circuitry having a plurality of recording channels, each recording channel being configured to receive signals from pixels of the multielectrode array.

18. The system of claim 18, wherein each recording channel of the recording circuitry comprises a reconfigurable component that is selectively controllable between a first mode, in which the reconfigurable component is configured to amplify a received pixel signal, and a second mode, in which the reconfigurable component is configured to selectively pass a frequency band of the received pixel signal.

19. The system of claim 15, further comprising a microfluidic system configured to deliver a first label to the cell culture or tissue and to flush the first label from the cell culture or tissue.

20. The system of claim 19, wherein the microfluidic system comprises a fluidic channel that is fluidically connected with the cell culture or tissue.
